(19) United States
(12) Patent Application Publication
(10) Pub. No.: US 2025/0011269 A1
(43) Pub. Date: Jan. 9, 2025

(54) CATALYTIC METHOD FOR PRODUCTION OF ACRYLIC ACID

(71) Applicant: CARGILL, INCORPORATED, Wayzata, MN (US)

(72) Inventors: Amandine CABIAC, Givors (FR); Vincent COUPARD, Lyon (FR); Souad RAFIK-CLEMENT, Vourles (FR); Justine TIRAPU, Brix (FR)

(73) Assignee: CARGILL, INCORPORATED, Wayzata, MN (US)

(21) Appl. No.: 18/712,341

(22) PCT Filed: Nov. 21, 2022
(86) PCT No.: PCT/US2022/080215

§ 371 Date: May 22, 2024

(30) Foreign Application Priority Data
Nov. 22, 2021 (FR) FR2112296

(51) Int. Cl. C07C 51/377; B01J 27/18; B01J 35/61; B01J 35/63; B01J 37/00; B01J 37/04; B01J 37/08; B01J 37/10

(52) U.S. Cl. CPC C07C 51/377; B01J 27/18; B01J 35/612; B01J 35/613; B01J 35/615; B01J 35/633; B01J 35/635; B01J 37/0036; B01J 37/04; B01J 37/088; B01J 37/10

(57) ABSTRACT

This disclosure relates to a novel material based on potassium phosphate salt or cesium phosphate salt and silica, comprising at least one source of silica formed into shape with at least one powder of a potassium phosphate or cesium phosphate salt and the use of this material for the preparation of acrylic acid. The disclosure also relates to a process for preparing said material, comprising at least one step of mixing at least one powder of at least one source of silica with at least one powder of at least one potassium phosphate or cesium phosphate salt and at least one solvent, a step of forming into shape preferably by extrusion of the mixture obtained on conclusion of the mixing step and a step of preparing calcined extrudates and optionally a final hydrothermal treatment step.

### CROSS-REFERENCE TO RELATED APPLICATIONS

This application claims the benefit of French Application No. FR2112296, filed Nov. 22, 2021, which is incorporated by reference herein in its entirety.

### TECHNICAL FIELD

The invention relates to the use of a novel material based on potassium phosphate salt or cesium phosphate salt and silica, comprising at least one source of silica formed into shape with at least one powder of a potassium phosphate or cesium phosphate salt. The novel material is useful as a catalyst for the conversion of lactic acid to acrylic acid. The invention also relates to a process for preparing said material, comprising at least one step of mixing at least one powder of at least one source of silica with at least one powder of at least one potassium phosphate or cesium phosphate salt and at least one solvent, a step of forming into shape preferably by extrusion of the mixture obtained on conclusion of the mixing step and a step of preparing calcined extrudates and optionally a final hydrothermal treatment step.

### BACKGROUND

It is known that silica, which is an advantageous compound for use as a catalytic support, cannot be extruded like other materials in conventional extrusion equipment to give products that are sufficiently resistant to be used in processes. This is because, from its manufacture to its use, the catalyst is confronted with numerous steps that can have an impact on its physical integrity. It must notably be resistant to crushing, to attrition and to pressure variations linked to the operating conditions of the catalytic reactor in which the process is performed. Thus, there is a continual need for catalysts which have improved mechanical and physical properties.

Patent application WO 2003/026795 relates to a process for producing a silica-supported catalyst, which consists in impregnating a silica constituent with a catalytic metal by means of an aqueous alkali bath, before drying in order to improve the mechanical strength thereof. More particularly, said process consists in forming and in washing a silica constituent, of silica gel or co-gel type, for example a silica-zirconia co-gel. Next, the washed silica constituent is brought into contact with the alkali bath in order for the impregnation with the catalytic metal, of cesium type, to take place, in order to form an activated silica constituent. The activated silica constituent is then dried in order to form a catalyst. The catalysts obtained have good mechanical strength. The present invention stands out through the route of production (by kneading-extrusion vs impregnation of silica beads) and the formulation of the solids obtained.

The WO 17/040383 (U.S. Pat. No. 9,849,447) patent family claims several generations of catalysts, all composed of a mixture of alkali metal phosphates, including some of formula MxPOy (M=K or Cs), and of a non-porous siliceous binder. The material described is prepared by mechanical mixing, by means of a planetary mill, of a dense amorphous molten silica (fused silica) material, which has no surface or porosity properties, and of potassium phosphate precursors. The catalyst calcined in air at 450° C. is composed of a KPO3/(KPO3+SiO2) mixture with a mass ratio of 13 to 26 wt % of KPO3. According to Example 8, the final catalyst is in the form of a powder having a variable screened particle size of between 106 and 212 μm.

The U.S. Pat. No. 8,884,050 patent family relates to the vapor phase dehydration of lactic acid to acrylic acid.

One object of the present invention is to provide a novel material comprising at least two sources of silica formed into shape with at least one powder of at least one potassium phosphate or cesium phosphate salt, said material having enhanced mechanical properties, notably in terms of mechanical strength.

Another object of the present invention is to provide a process for preparing said material according to the invention, said material obtained having good mechanical strength and in particular a high SPC suitable for its use in the presence of a solvent and thus in an industrial process over long periods, notably by means of the use of a hydrothermal treatment step in a preferred embodiment of the invention.

Another object of the present invention is to provide a material formed into shape which can be used as a catalytic support or as a catalyst in catalytic processes to convert lactic acid to acrylic acid.

### SUMMARY

More precisely, the present invention relates to a process for the preparation of acrylic acid comprising the steps of: i) preparing a lactic acid containing feedstock, ii) heating the feedstock to obtain a lactic acid containing vapor phase, iii) contacting the lactic acid containing vapor phase with a catalyst to create a reacted vapor phase, and iv) isolating acrylic acid from the reacted vapor phase wherein the catalyst is prepared by a process, comprising at least the following steps:
a) a step of mixing at least one precipitated silica powder and at least one colloidal silica sol with at least one powder of at least one potassium phosphate salt and/or at least one cesium phosphate salt in at least one solvent to obtain a mixture,
b) a step of forming into shape of the mixture obtained on conclusion of step a),
c) a step of maturation of the material obtained on conclusion of step b).

The present invention also relates to the material formed into shape obtained by said process, said material comprising a source of precipitated silica, a colloidal silica sol and at least one potassium phosphate and/or cesium phosphate salt, said material advantageously having a ratio MPO3/(MPO3+SiO2) of between 20 and 70, preferably between 22 and 70 with M=K or Cs.

One advantage of the present invention is that of providing a process for preparing a material based on potassium phosphate salt or cesium phosphate salt and silica, having improved mechanical strength relative to the materials of the prior art by means of performing a step of mixing two specific precursors of silica and of potassium and/or cesium phosphate salt, followed by the forming into shape of the mixture and then maturation of the extruded objects obtained, preferably combined with performing a final step of hydrothermal treatment of the material obtained.

One advantage of the present invention is that of providing a process for preparing a material comprising a source of precipitated silica, a colloidal silica sol and at least one potassium phosphate and/or cesium phosphate salt, which has mechanical strength that is particularly improved relative to the materials of the prior art by means of the use in step a) of a mixture of a source of precipitated silica or silica gel having a reduced size and preferably less than 10 μm, preferably less than 5 μm, more preferably less than 1 μm, combined with the use of a powder of at least one potassium and/or cesium phosphate salt that are milled and screened to a particle size of less than 100 μm.

Another object of the present invention is that of producing a material formed into shape which can be used as a catalytic support or as a catalyst in catalytic processes.

Another object is a process for the preparation of acrylic acid comprising the steps of: i) preparing a lactic acid containing feedstock, heating the feedstock to obtain a lactic acid containing vapor phase, contacting the lactic acid containing vapor phase with a catalyst to create a reacted vapor phase and isolating acrylic acid from the reacted vapor phase where in the catalyst is prepared by the methods disclosed herein.

### DETAILED DESCRIPTION

For the purposes of the present invention, the various embodiments presented may be used alone or in combination with each other, without any limit to the combinations.

For the purposes of the present invention, the various ranges of parameters for a given step, such as the pressure ranges and the temperature ranges, may be used alone or in combination. For example, for the purposes of the present invention, a preferred range of pressure values can be combined with a more preferred range of temperature values.

Throughout the text hereinbelow; the term “side crush strength” means the mechanical strength of the material according to the invention, determined by the single pellet crush (SPC) test. This is a standardized test (standard ASTM D4179-01) which consists in subjecting a material in the form of a millimetre-sized object, such as a bead, a pellet or an extruded object, to a compressive force generating break. This test is thus a measurement of the tensile strength of the material. The analysis is repeated on a certain number of solids taken individually and typically on a number of solids of between 10 and 200. The mean of the breaking side forces measured constitutes the mean SPC which is expressed, in the case of granules, in unit force (N), and in the case of extruded objects, in unit force per unit length (daN/mm or decaNewtons per millimetre of extruded object length).

Throughout the text hereinbelow, the term “specific surface area” means the BET specific surface area (SBET) determined by nitrogen adsorption in accordance with the standard ASTM D 3663-78 established from the Brunauer-Emmett-Teller method described in the journal The Journal of the American Chemical Society, 60, 309 (1938).

The term “macropores” means pores whose aperture is greater than 50 nm.

The term “mesopores” means pores whose aperture is between 2 nm and 50 nm, limits inclusive.

The term “total pore volume” (TPV) of the material according to the invention means the volume measured by mercury intrusion porosimetry according to the standard ASTM D4284-83 at a maximum pressure of 4000 bar (400 MPa), using a surface tension of 484 dynes/cm and a contact angle of 140°. The wetting angle was taken equal to 140° following the recommendations of the publication “Techniques de l′ingénieur, traité analyse et caractérisation” [Techniques of the Engineer, Analysis and Characterization Treatise], pages 1050-1055, written by Jean Charpin and Bernard Rasneur.

In order to obtain better accuracy, the value of the total pore volume corresponds to the value of the total pore volume measured by mercury intrusion porosimetry measured on the sample minus the value of the total pore volume measured by mercury intrusion porosimetry measured on the same sample for a pressure corresponding to 30 psi (about 0.2 MPa).

The volume of the macropores and of the mesopores is measured by mercury intrusion porosimetry according to the standard ASTM D4284-83 at a maximum pressure of 4000 bar (400 MPa), using a surface tension of 484 dynes/cm and a contact angle of 140°. The value from which the mercury fills all the intergranular voids is set at 0.2 MPa and it is considered that, above this, the mercury penetrates into the pores of the sample.

The macropore volume of the material according to the invention is defined as being the cumulative volume of mercury introduced at a pressure of between 0.2 MPa and 30 MPa, corresponding to the volume contained in the pores with an apparent diameter of greater than 50 nm.

The mesopore volume of the material according to the invention is defined as being the cumulative volume of mercury introduced at a pressure of between 30 MPa and 400 MPa, corresponding to the volume contained in the pores with an apparent diameter of between 2 and 50 nm.

The median diameter of the macropores (Dmacro in nm) is also defined as being a diameter such that all the pores of size less than this diameter constitute 50% of the mesopore volume, measured by mercury porosimetry.

In the text hereinbelow, transmission electron microscopy (TEM) is the method used to characterize the materials obtained according to the invention. This technique makes it possible to obtain information on the chemical composition and the morphology, and to measure the size of the grains or of the crystals constituting the material. For that, use is made of an electron microscope (of the JEOL JEM F200 or JEOL JEM 2100F type) equipped with an energy dispersive spectrometer (EDS). The EDS detector must allow the light elements to be detected. The combination of these two tools, TEM and EDS, makes it possible to combine the imaging and local chemical analysis with good spatial resolution.

In the text hereinbelow, the size of the grains or particle size of the constituents of the materials obtained according to the invention is measured by the laser scattering particle size analysis technique. This indirect measurement technique makes it possible to determine the size distribution of particles (scale of 1 micron to 1 millimetre). This analysis method uses the principle of light scattering (Mie theory) and/or of light diffraction (Fraunhofer theory and Mie theory). The particles illuminated by the laser light deviate the light from its main axis. The amount of light deviated and the size of the angle of deviation make it possible to accurately measure the particle size. The powder is conveyed either by a solvent (water, isopropanol) or by air before passing in front of the laser beam: two approaches are thus distinguished: wet particle size analysis and dry particle size analysis.

Wet particle size analysis makes it possible to characterize dispersions (elementary particle size analysis after dispersion) or solids in suspension (“aggregated” particle size analysis). The particles measured are in the range 0.02 to 2000 microns.

Dry particle size analysis makes it possible to characterize powders of which the initial aggregation is not destroyed. The measurement range extends from 0.2 to 2000 microns. In the present invention, dry particle size analysis is used to measure the size of the grains of the constituents of the material of the invention.

In accordance with the invention, the present invention relates to a process for preparing a material, comprising at least the following steps:
a) a step of mixing at least one precipitated silica powder and at least one colloidal silica sol with at least one powder of at least one potassium phosphate salt and/or at least one cesium salt in at least one solvent to obtain a mixture,
b) a step of forming into shape of the mixture obtained on conclusion of step a),
c) a step of maturation of the material obtained on conclusion of step b).

### Step a):

In accordance with the invention, said step a) consists in mixing at least one precipitated silica powder with at least one colloidal silica sol and at least one powder of at least one potassium phosphate salt and/or at least one cesium salt in at least one solvent to obtain a mixture.

Preferably, the precipitated silica powder or silica gels are chosen, without being restricted, from the following commercial sources: Nyasil20 (Nyacol®), Siliaflash P60 (Silicycle®), Siliaflash C60 (Silicycle®), Ultrasil VN3 GR (Evonik®), taken alone or as a mixture.

Preferably, the precipitated silica powder or silica gel has a grain size of less than 10 μm, preferably less than 5 μm and more preferably less than 1 μm.

Preferably, the colloidal silica sols are chosen, without being restricted, from the following commercial sources: Ludox (W.R. Grace Davison®), Nyacol (Nyacol Nano Technologies®, Inc. or PQ Corp®), Nalco (Nalco Chemical Company®), Ultra-Sol (RESI Inc®), NexSil (NNTI®), taken alone or as a mixture.

The majority of the colloidal silica sols are prepared from sodium silicate and inevitably contain sodium. Since the presence of sodium may prove to be detrimental to the catalytic activity, an ion-exchange step may be necessary in order to reduce or even eliminate the residual sodium. In order to dispense with this step, the use of low-sodium colloidal silica sols is preferable: by way of example, mention may be made of Ludox AS40 stabilized with an ammonium counterion or Nyacol 2034DI, Nalco 1034A, Ultra-Sol 7H or NexSil 20A.

Said sources of silica(s) used in the process going to the present invention are advantageously amorphous synthetic silicas.

Said potassium phosphate salt(s) and/or cesium phosphate salt(s) used in step a) are advantageously chosen from potassium phosphate salts or cesium phosphate salts in amorphous or crystalline oxide form, taken alone as a mixture.

Said potassium phosphate salt(s) are advantageously chosen from the following list: KH2PO4, KH2P2012, K6P607, K3H2P3010, K4H2P4013, K3P309, K4P4012, K6P6018, K8P8024, K10P10030, potassium (tripotassium) phosphate (3K+ PO43−), alone or as a mixture. Preferably, the preferred potassium phosphate salt is chosen from potassium (tripotassium) phosphate (3K+ PO43−) and KH2PO4, alone or as a mixture.

Said cesium phosphate salt(s) are advantageously chosen from the following list: CsH2PO4, Cs2H2P3010, Cs4H2P4013, Cs3P309, Cs4P4012, Cs6P6018, Cs8P8024, (CsPO3), alone or as a mixture. Preferably, the preferred cesium phosphate salt is Cs2HPO4.

Preferably, said potassium phosphate salt(s) or cesium phosphate salt(s) are chosen from potassium (tripotassium) phosphate (3K+ PO43−), KH2PO4, CsH2PO4 optionally in their hydrated form.

Preferably, at least one organic adjuvant is also mixed in during step a).

Said organic adjuvant may also be chosen from all the additives known to those skilled in the art.

In the case where at least one organic adjuvant is added in step a), said organic adjuvant is advantageously chosen from cellulose derivatives, polyethylene glycols, aliphatic monocarboxylic acids, aromatic alkylated compounds, sulfonic acid salts, fatty acids, polyvinylpyrrolidone, polyvinyl alcohol, methylcellulose, polyacrylates, polymethacrylates, polyisobutene, polytetrahydrofuran, starch, polymers of polysaccharide type (such as xanthan gum), scleroglucan, derivatives of hydroxyethylcellulose or carboxymethylcellulose type, lignosulfonates and galactomannan derivatives, taken alone as a mixture.

Preferably, said organic adjuvant may be mixed in powder form or in solution in said solvent.

Said solvent is advantageously chosen from water, ethanol, alcohols and amines. Preferably, said solvent is water.

In the context of the invention, it is entirely possible to envisage mixing together several different silica powders and/or different silica sols and/or different powders of potassium phosphate or cesium phosphate.

The order in which the mixing of the powders of at least the sources of silica, of at least one powder of potassium and/or cesium phosphate salt and optionally of at least one organic adjuvant, in the case where they are mixed in powder form, with at least one solvent is performed is irrelevant.

The mixing of said powders and of said solvent may advantageously be performed a single time.

The additions of powders and of solvent may also be advantageously alternated.

Said potassium phosphate and/or cesium phosphate salt(s) used in step a) are advantageously in the form of powders.

Preferably, said potassium and/or cesium phosphate salt(s), in the case where they are mixed in powder form, may advantageously be milled and screened to a particle size of less than 100 μm.

Preferably, the source of precipitated silica or silica gel used in step a) has a grain size of less than 10 μm, preferably less than 5 μm and more preferably less than 1 μm.

In a particularly preferred embodiment, the use of a source of precipitated silica or silica gel having a reduced grain size preferably less than 10 μm, preferably less than 5 μm, more preferably less than 1 μm, combined with the use of a powder of at least one potassium and/or cesium phosphate salt which are milled and screened to a grain size of less than 100 μm, allows a significant improvement in the mechanical strength of the materials obtained according to the invention.

Preferably, said powders of at least one silica, of at least one potassium and/or cesium phosphate salt and optionally of at least one organic adjuvant, in the case where they are mixed in powder form, are first dry-premixed, before the introduction of the solvent.

Said premixed powders are then advantageously placed in contact with said solvent. In another embodiment, at least said sources of silica and at least said organic adjuvant may be in solution or suspension beforehand in said solvent when said solvent is placed in contact with the potassium and/or cesium phosphate powders. The placing in contact with said solvent leads to the production of a mixture which is then kneaded.

In the case where at least one powder of cesium phosphate salt is used, the addition of ammonia may be necessary to obtain an extrudable mixture.

Preferably, said mixing step a) is performed by batchwise or continuous kneading.

In the case where said step a) is performed batchwise, said step a) is advantageously performed in a kneader preferably equipped with Z-shaped arms, or a cam mixer, or in any other type of mixer, for instance a planetary mixer. Said mixing step a) makes it possible to obtain a homogeneous mixture of the pulverulent constituents.

Preferably, said step a) is performed at room temperature, for a time of between 5 and 60 minutes, and preferably between 10 and 50 minutes. The rotation speed of the kneading arms is advantageously between 10 and 75 rpm, preferably between 25 and 50 rpm.

Preferably, the following amounts are introduced in the mixing step a) of the process according to the invention:
1% to 99% by weight, preferably from 5% to 99% by weight, preferably from 10% to 95% by weight, and very preferably from 15% to 65% by weight of at least one precipitated silica,
1% to 99% by weight, preferably from 5% to 99% by weight, preferably from 10% to 95% by weight, and very preferably from 5% to 50% by weight of at least one colloidal silica sol,
1% to 99% by weight, preferably from 5% to 99% by weight, preferably from 10% to 95% by weight, and very preferably from 20% to 75% by weight of at least one powder of a potassium or cesium phosphate salt,
0% to 20% by weight, preferably from 1% to 15% by weight, preferably from 1% to 10% by weight, and very preferably from 1% to 7% by weight of at least one organic adjuvant, in the case where said material is not calcined, the weight percentages being expressed relative to the total weight of said material, and the sum of the contents of each of the compounds of said material being equal to 100%.

### Step b):

In accordance with the invention, said step b) consists in forming into shape the mixture obtained on conclusion of step a).

Preferably, the mixture obtained on conclusion of step a) is advantageously formed into shape by extrusion.

When the forming into shape of the mixture resulting from step a) is performed by extrusion, said step b) is advantageously performed in a single-screw or twin-screw piston extruder.

In this case, an organic adjuvant may optionally be added in the mixing step a). The presence of said organic adjuvant facilitates the forming into shape by extrusion. Said organic adjuvant is described above and is introduced in step a) in the proportions indicated above.

In the case where said preparation process is performed continuously, said mixing step a) may be coupled with step b) of forming into shape by extrusion in the same equipment. According to this embodiment, the extrusion of the mixture, also called the “kneaded paste”, may be performed either by directly extruding the end of a twin-screw continuous kneader for example, or by connecting one or more batch kneaders to an extruder. The geometry of the die, which gives the extrudates their shape, can be chosen from dies well known to those skilled in the art. They may thus, for example, be of cylindrical, multilobal, fluted or slotted shape.

In the case where the forming into shape of the mixture resulting from step a) is performed by extrusion, the amount of solvent added in the mixing step a) is adjusted so as to obtain, on conclusion of this step and regardless of the variant implemented, a mixture or a paste which does not run but which is also not too dry, so as to allow its extrusion under suitable pressure conditions well known to those skilled in the art and dependent on the extrusion equipment used.

Preferably, said step b) of forming into shape by extrusion is performed at an extrusion pressure greater than 1 MPa and preferably between 3 MPa and 10 MPa.

### Step c):

The process for preparing said material according to the invention comprises a step c) of maturing the material formed into shape, obtained on conclusion of step b). Said maturation step is advantageously performed at a temperature of between 0° C. and 300° C., preferably between 20° C. and 200° C. and preferably between 2° and 150° C., for a time of between 1 minute and 72 hours, preferably between 30 minutes and 72 hours, preferably between 1 hour and 48 hours and more preferably between 1 and 24 hours.

Preferably, said maturation step is performed in air and preferably in moist air with a relative humidity of between 20% and 100% and preferably between 70% and 100%. This step allows good hydration of the material required to limit the appearance of cracks which are harmful to the mechanical strength.

Advantageously, the material formed into shape and resulting from the maturation step c) may also optionally undergo a calcining step c′) at a temperature of between 50° and 800° C., preferably between 100 and 550° C. for a period of between 1 and 12 hours and preferably between 1 and 4 hours. This calcining step is notably useful in order to eliminate the organic adjuvants used so as to facilitate the forming into shape of the material.

Said optional calcining step c′) is advantageously performed under a gas stream comprising oxygen: for example, the extrudates are preferably calcined in dry air or with various degrees of humidity or else heat-treated in the presence of a gas mixture comprising an inert gas, preferably nitrogen, and oxygen. The gas mixture used preferably comprises at least 5% by volume or even preferably at least 10% by volume of oxygen.

### Optional Step d):

In a preferred embodiment, the process comprises a step d) of hydrothermal treatment in the presence of steam.

The hydrothermal treatment is performed via any technique known to those skilled in the art. The term “hydrothermal treatment” means the placing in contact, at any step in the production, of the mixed support with water in the vapour phase or in the liquid phase. The term “hydrothermal treatment” may notably mean maturing, steaming, autoclaving, calcining in moist air, or rehydration. Without this reducing the scope of the invention, such a treatment has the effect of making the silica component mobile.

Said step d) may advantageously be performed at atmospheric pressure or under a partial pressure of water.

In a very preferred embodiment, the hydrothermal treatment step d) is performed at atmospheric pressure, at a temperature of between 200 and 1100° C. and preferably of between 400° C. and 1000° C., for a time of between 30 minutes and 5 hours, the composition by volume of water in the gas in said step d) being between 5% and 100%, preferably between 10% and 90%.

In another very preferred embodiment, the hydrothermal treatment step d) is performed under a partial pressure of water. The support may thus be advantageously subjected to a hydrothermal treatment in a confined atmosphere or by autoclaving. The term “hydrothermal treatment in a confined atmosphere” means a treatment by passing through an autoclave in the presence of water at a temperature above room temperature.

According to this very preferred embodiment of the invention, the hydrothermal treatment is a heat treatment under a stream containing gaseous water and a gas, and under pressure. The gas is advantageously air or nitrogen. The composition by volume of the water in the gas is advantageously between 5% and 100%, preferably between 10% and 90%. The temperature during the hydrothermal treatment may be between 100 and 1100° C., preferably between 100 and 550° C. and preferably between 200° C. and 450° C., for a time of between 30 minutes and 24 hours and preferably between 30 minutes and 4 hours. The partial pressure of water is between 0.1 and 10 MPa, preferably between 0.11 and 7.5 MPa and more preferably between 0.1 and 5 MPa.

In a preferred embodiment, said hydrothermal treatment step d) may, where appropriate, totally or partly replace the calcining step c′).

On conclusion of the process for preparing the material according to the invention, the material obtained is in the form of extrudates or of pellets.

However, it is not excluded for said materials obtained to subsequently be, for example, introduced into equipment which allows their surface to be rounded off, such as a pan or any other equipment allowing the spheronization of said materials.

Said preparation process according to the invention makes it possible to obtain materials according to the invention having mechanical strength values measured by single pellet crush of greater than or equal to 0.2 daN/mm, preferably greater than or equal to 0.4 daN/mm, preferably greater than or equal to 0.6 daN/mm and preferably greater or equal than 0.8 daN/mm.

The material obtained on conclusion of the preparation process according to the invention may be used for applications in catalysis.

Said material is brought into contact with the gaseous feedstock to be treated in a reactor, which may be either a fixed-bed reactor, or a radial reactor, or else a fluidized-bed reactor.

Another subject of the present invention relates to the material that may be obtained via said process according to the invention.

Another subject of the present invention relates to a material comprising at least one source of precipitated silica, at least one colloidal silica sol, formed into shape with at least one powder of potassium phosphate salt and/or of cesium phosphate salt.

Said source of precipitated silica and the colloidal silica sol are defined above.

Transmission electron microscopy (TEM) makes it possible to reveal the presence of the two different sources of silica on the final material and also the distribution of the various constituents of said material.

Preferably, said material has the following composition:
1% to 99% by weight, preferably from 5% to 99% by weight, preferably from 10% to 95% by weight, and very preferably from 15% to 65% by weight of at least one precipitated silica,
1% to 99% by weight, preferably from 5% to 99% by weight, preferably from 10% to 95% by weight, and very preferably from 5% to 50% by weight of at least one colloidal silica sol,
1% to 99% by weight, preferably from 5% to 99% by weight, preferably from 10% to 95% by weight and very preferably from 20% to 75% by weight of at least one potassium phosphate or cesium phosphate salt,
0% to 20% by weight, preferably from 1% to 15% by weight, preferably from 1% to 10% by weight, and very preferably from 1% to 7% by weight of at least one organic adjuvant, in the case where said material is not calcined, the weight percentages being expressed relative to the total weight of said material, and the sum of the contents of each of the compounds of said material being equal to 100%.

The mass percentages are expressed relative to the anhydrous mass of the final composite material. This anhydrous mass is determined by measuring said loss on ignition corresponding to the mass variation resulting from heating the sample at 1000° C. for 2 hours. The loss on ignition is expressed as a mass percentage of the solids.

Preferably, said material has a mass percentage MPO3/(MPO3+SiO2) of between 20 and 70, preferably between 22 and 70 with M=K or Cs.

Said material according to the present invention is advantageously in the form of extrudates, beads or pellets.

Said materials according to the invention have specific surface areas of between 0 and 240 m²/g.

Said materials according to the invention have a total pore volume of between 0 and 0.7 cm3/g.

Said materials according to the invention have a macropore volume of between 0 and 0.30 cm3/g.

Said materials according to the invention have a mesopore volume of between 0 and 0.4 cm3/g.

Said materials according to the invention have enhanced mechanical properties, notably in terms of mechanical strength, without premilling the precursors for potassium phosphate salt contents of greater than 45% by weight and greater than or equal to 35% by weight for the cesium phosphate salt.

In a preferred embodiment, the powders of potassium phosphate and/or cesium phosphate salt are premilled and screened before mixing with the sources of silica so that the particle size of the potassium phosphate and/or cesium phosphate salts is less than 100 microns. The grain size of the potassium phosphate and/or cesium phosphate salts may advantageously be controlled by dry laser particle size analysis.

In the case where powders of potassium phosphate and/or cesium phosphate salt are premilled and screened, said materials obtained according to the invention have enhanced mechanical properties, notably in terms of mechanical strength with premilling of the potassium phosphate and cesium phosphate precursors, for contents of potassium phosphate salt of less than 45% by weight and less than 35% by weight for the cesium phosphate salt. Premilling of the powders makes it possible to obtain a material that is more homogeneous and more resistant with respect to the presence of water or of solvent.

Increasing the mechanical strength makes it possible to envisage using said material in processes in the presence of water or of solvents and at relatively high temperatures.

Said materials according to the invention may thus be used for applications in catalysis and separation.

In particular, said materials according to the invention have a mechanical strength measured by the single pellet crush test, referred to hereinbelow as SPC, at least greater than or equal to 0.4 daN/mm, preferably at least greater than or equal to 0.6 daN/mm and preferably at least greater than or equal to 0.8 daN/mm.

### Catalytic Preparation of Acrylic Acid

One aspect included is a process for the preparation of acrylic acid comprising the steps of: i) preparing a lactic acid containing feedstock, ii) heating the feedstock to obtain a lactic acid containing vapor phase, iii) contacting the lactic acid containing vapor phase with a catalyst to create a reacted vapor phase, and iv) isolating acrylic acid from the reacted vapor phase where in the catalyst is prepared by methods disclosed herein.

A “lactic acid containing feedstock” as used herein means a solution or mixture of components containing lactic acid. The lactic acid may be included in any concentration. In some aspects the lactic acid containing feedstock is an aqueous solution. In some aspects lactic acid containing feedstock is an aqueous solution containing between 5% and 25% by weight lactic acid. In some aspects lactic acid containing feedstock is an aqueous solution containing between 15% and 85% by weight lactic acid. In some aspects lactic acid containing feedstock is an aqueous solution containing between 50% and 75% by weight lactic acid.

The lactic acid containing feedstock may then be heated in reactor to vaporization. The lactic acid containing feedstock may be dripped or injected into the preheated reactor where it will quickly vaporize. Lactic acid vaporization is known in the art and any suitable reactor or series of reactors may be utilized in the present invention. The reaction may be carried out at a temperature of between 350° C. and 400° C. and at an elevated pressure. The reaction may include the use of a carrier gas such as nitrogen or steam. The carrier gas may be added externally or prepared by the vaporization of the feedstock, or both.

The vaporized feedstock is then contacted with the catalysts of the present disclosure. These catalysts are shown to efficiently facilitate the dehydration of lactic acid to acrylic acid.

After conversion, the reaction gases are cooled, acrylic acid is condensed and isolated as the product of the reaction. The condensation and isolation of the acrylic acid product is also a well-known procedure in the art.

### EXAMPLES

The examples below illustrate the invention without limiting the scope thereof.

### Example 1

In order to illustrate the invention, several preparation methods are described, on the basis of the forming into shape of a silica, in particular of a precipitated silica and of a colloidal silica sol (Ludox AS40 (W.R. Grace Davison®)). The contents are expressed as mass percentages.

### Example 1 According to the Invention: Preparation without a Milling Step of the Powder of Potassium Phosphate Salt and without a Hydrothermal Treatment Step

A precipitated silica powder (Siliaflash C60 5-20 μm: Silicycle) (35.5%), a source of colloidal silica sol (35.5%), potassium dihydrogen phosphate (KH2PO4: Aldrich) (29%) and Methocel (K15M) (3%) are introduced into and premixed in a Brabender kneader. Water is added dropwise until a paste is obtained and the kneading is continued for 20 minutes. The paste obtained is then extruded on an MTS piston extruder using a cylindrical die with a diameter of 1.6 mm.

The extrudates are matured for 16 hours at 80° C. in a ventilated oven and then calcined for 4 hours at 450° C.

The extrudates obtained have an SPC value of 0.28 daN/mm, an SBET of 103 m²/g, a TPV of 0.52 cm3/g with a macropore volume of 0.19 cm3/g, a mesopore volume of 0.25 cm3/g and a Dmacro=1099 nm. The material obtained has a mass ratio KPO3/(KPO3+SiO2)=26.2%.

### Example 2 According to the Invention: Preparation with a Milling Step of the Powder of Potassium Phosphate Salt and without a Hydrothermal Treatment Step

A precipitated silica powder (Siliaflash C60 5-20 μm: Silicycle) (35.5%), a source of colloidal silica sol (35.5%), potassium dihydrogen phosphate (KH2PO4: Aldrich) milled and screened to 100 μm (29%) and Methocel (K15M) (3%) are introduced into and premixed in a Brabender kneader. Water is added dropwise until a paste is obtained and the kneading is continued for 20 minutes. The paste obtained is then extruded on an MTS piston extruder using a cylindrical die with a diameter of 1.6 mm.

The extrudates are matured for 16 hours at 80° C. in a ventilated oven and then calcined for 4 hours at 450° C.

The extrudates obtained have an SPC value of 0.4 daN/mm, an SBET of 96 m²/g, a TPV of 0.46 cm3/g with a macropore volume of 0.17 cm3/g, a mesopore volume of 0.25 cm3/g and a Dmacro=679 nm. The material obtained has a mass ratio KPO3/(KPO3+SiO2)=26.2%.

### Example 3 According to the Invention: Preparation without a Milling Step of the Powder of Potassium Phosphate Salt but with Introduction of a High Content of Potassium Phosphate Salt and without a Hydrothermal Treatment Step

A precipitated silica powder (Siliaflash C60 5-20 μm: Silicycle) (15%), a source of colloidal silica sol (15%), potassium dihydrogen phosphate (KH2PO4: Aldrich) (70%) and Methocel (K15M) (3%) are introduced into and premixed in a Brabender kneader. Water is added dropwise until a paste is obtained and the kneading is continued for 20 minutes. The paste obtained is then extruded on an MTS piston extruder using a cylindrical die with a diameter of 1.6 mm.

The extrudates are matured for 16 hours at 80° C. in a ventilated oven and then calcined for 4 hours at 450° C.

The extrudates obtained have an SPC value of 0.9 daN/mm, an SBET of 2 m²/g, a TPV of 0.32 cm3/g with a macropore volume of 0.17 cm3/g, a mesopore volume of 0.12 cm3/g and a Dmacro=1836 nm. The material obtained has a mass ratio KPO3/(KPO3+SiO2)=67%.

### Example 4 According to the Invention: Preparation without a Milling Step of the Powder of Cesium Phosphate Salt and without a Hydrothermal Treatment Step

A precipitated silica powder (Nyasil20 1.5 μm: Nyacol) (56%), a source of colloidal silica sol (9%), cesium dihydrogen phosphate (CsH2PO4: Alfa Chemistry) (35%) and Methocel (K15M) (3%) are introduced into and premixed in a Brabender kneader. Water supplemented with aqueous ammonia is added dropwise until a paste is obtained and kneading is continued for 20 minutes. The paste obtained is then extruded on an MTS piston extruder using a cylindrical die with a diameter of 1.6 mm.

The extrudates are matured for 16 hours at 80° C. in a ventilated oven and then calcined for 12 hours at 450° C.

The extrudates obtained have an SPC value of 0.8 daN/mm, an SBET of 22 m²/g, a TPV of 0.32 cm3/g with a macropore volume of 0.01 cm3/g, a mesopore volume of 0.30 cm3/g and a Dmacro=806 nm. The material obtained has a ratio CsPO3/(CsPO3+SiO2)=33.2%.

### Example 5 According to the Invention: Preparation with a Milling Step of the Potassium Phosphate Salt and Use of a Source of Precipitated Silica with a Low Particle Size and without a Hydrothermal Treatment Step

A precipitated silica powder (Nyasil20 1.5 μm: Nyacol) (64.31%), a source of colloidal silica sol (10.97%), potassium dihydrogen phosphate (KH2PO4; Aldrich) milled and screened to 100 μm (24.73%) and Methocel (K15M) (3%) are introduced into and premixed in a Brabender kneader. Water is added dropwise until a paste is obtained and the kneading is continued for 20 minutes. The paste obtained is then extruded on an MTS piston extruder using a cylindrical die with a diameter of 1.6 mm.

The extrudates are matured for 16 hours at 80° C. in a ventilated oven and then calcined for 4 hours at 450° C.

The extrudates obtained have an SPC value of 0.7 daN/mm and an SBET of 56 m²/g, a TPV of 0.33 cm3/g with a macropore volume of 0.14 cm3/g, a mesopore volume of 0.15 cm3/g and a Dmacro=351 nm. The material obtained has a ratio KPO3/(KPO3+SiO2)=26.2%.

### Example 6 According to the Invention: Preparation with a Milling Step of the Potassium Phosphate Salt and Use of a Source of Precipitated Silica with a Low Particle Size and with a Hydrothermal Treatment Step at Atmospheric Pressure

A precipitated silica powder (Nyasil20 1.5 μm: Nyacol) (64.31%), a source of colloidal silica sol (10.97%), potassium dihydrogen phosphate (KH2PO4; Aldrich) milled and screened to 100 μm (24.73%) and Methocel (K15M) (3%) are introduced into and premixed in a Brabender kneader. Water is added dropwise until a paste is obtained and the kneading is continued for 20 minutes. The paste obtained is then extruded on an MTS piston extruder using a cylindrical die with a diameter of 1.6 mm.

The extrudates are matured for 16 hours at 80° C. in a ventilated oven and are then subjected to a hydrothermal treatment step at atmospheric pressure for 3 hours at a temperature of 500° C. The composition by volume of water in the air for the hydrothermal treatment is 50%.

The extrudates obtained have an SPC value of 1.05 daN/mm and an SBET of 37 m²/g, a TPV of 0.38 cm3/g with a macropore volume of 0.18 cm3/g, a mesopore volume of 0.16 cm3/g and a Dmacro=310 nm. The material obtained has a ratio KPO3/(KPO3+SiO2)=26.2%.

### Example 7 According to the Invention: Preparation with a Milling Step of the Potassium Phosphate Salt and Use of a Source of Precipitated Silica with a Low Particle Size and with a Hydrothermal Treatment Step Under Pressure

A precipitated silica powder (Nyasil20 1.5 μm: Nyacol) (64.31%), a source of colloidal silica sol (10.97%), potassium dihydrogen phosphate (KH2PO4; Aldrich) milled and screened to 100 μm (24.73%) and Methocel (K15M) (3%) are introduced into and premixed in a Brabender kneader. Water is added dropwise until a paste is obtained and the kneading is continued for 20 minutes. The paste obtained is then extruded on an MTS piston extruder using a cylindrical die with a diameter of 1.6 mm.

The extrudates are matured for 16 hours at 80° C. in a ventilated oven and then subjected to a hydrothermal treatment step under pressure. The partial pressure of water is set at 0.42 MPa and the temperature is set at 375° C. for 1 hour.

The composition by volume of water in the air for the hydrothermal treatment is 50%.

The extrudates obtained have an SPC value of 2.81 daN/mm and an SBET of 21 m²/g, a TPV of 0.25 cm3/g with a macropore volume of 0.12 cm3/g, a mesopore volume of 0.07 cm3/g and a Dmacro=307 nm. The material obtained has a ratio KPO3/(KPO3+SiO2)=26.2%.

### Example 8 According to the Invention: Preparation Process without a Milling Step Using Potassium (Tripotassium) Phosphate (3K+ PO43−), with a Hydrothermal Treatment Step at Atmospheric Pressure

A precipitated silica powder (Nyasil20: Nyacol) (36.95%), a source of colloidal silica sol (36.95%), potassium phosphate (KPO3: Aldrich) (26.1%) and Methocel (K15M) (3%) are introduced into and premixed in a Brabender kneader. Water is added dropwise until a paste is obtained and the kneading is continued for 20 minutes. The paste obtained is then extruded on an MTS piston extruder using a cylindrical die with a diameter of 1.6 mm.

The extrudates are dried for 16 hours at 120° C. in a ventilated oven and are then subjected to a hydrothermal treatment step at atmospheric pressure for 3 hours at a temperature of 500° C. The composition by volume of water in the air for the hydrothermal treatment is 50%.

The extrudates obtained have an SPC value of 0.85 daN/mm and an SBET of 60 m²/g, a TPV of 0.34 cm3/g with a macropore volume of 0.06 cm3/g, a mesopore volume of 0.25 cm3/g and a Dmacro=260 nm. The material obtained has a ratio KPO3/(KPO3+SiO2)=26.2%.

### Example 9 According to the Invention: Preparation Process without a Milling Step Using Potassium (Tripotassium) Phosphate (3K+ PO43−), with a Hydrothermal Treatment Step at Atmospheric Pressure

A precipitated silica powder (Nyasil20); Nyacol) (30%), a source of colloidal silica sol (30%), potassium phosphate (KPO3: Aldrich) (40%) and Methocel (K15M) (3%) are introduced into and premixed in a Brabender kneader. Water is added dropwise until a paste is obtained and the kneading is continued for 20 minutes. The paste obtained is then extruded on an MTS piston extruder using a cylindrical die with a diameter of 1.6 mm.

The extrudates are dried for 16 hours at 80° C. in a ventilated oven and are then subjected to a hydrothermal treatment step at atmospheric pressure for 3 hours at a temperature of 500° C. The composition by volume of water in the air for the hydrothermal treatment is 50%.

The extrudates obtained have an SPC value of 1 daN/mm and an SBET of 43 m²/g, a TPV of 0.44 cm3/g with a macropore volume of 0.18 cm3/g, a mesopore volume of 0.23 cm3/g and a Dmacro=706 nm. The material obtained has a ratio KPO3/(KPO3+SiO2)=40%.

### Comparative Example 10: Mixing Step a) Using a Single Silica Precursor, Fused Silica, and Two Phosphate Salts Followed by a Test of Forming into Shape by Extrusion

A powder of fused silica (Sigma-Aldrich milled and screened to between 102-212 μm) (68.3%), potassium hydrogen phosphate (K2HPO4: Aldrich) (18.1%), diammonium phosphate (13.6%) ((NH4)2HPO4: Aldrich) and Methocel (K15M) (3%) are introduced into and premixed in a Brabender kneader. Water is added dropwise so as to allow a paste to be obtained. This formulation does not make it possible to obtain an extrudable paste.

### Comparative Example 11: Mixing Step a) Using a Single Silica Precursor, a Precipitated Silica and Two Phosphate Salts Followed by a Test of Forming into Shape by Extrusion

A powder of precipitated silica (5-20 μm) (Siliaflash: Silicycle) (68.6%), potassium hydrogen phosphate (K2HPO4: Aldrich) (17.8%), diammonium phosphate (13.6%) ((NH4)2HPO4: Aldrich) and Methocel (K15M) (3%) are introduced into and premixed in a Brabender kneader. Water is added dropwise until a paste is obtained and the kneading is continued for 20 minutes. The paste obtained is then extruded on an MTS piston extruder using a cylindrical die with a diameter of 1.6 mm.

The extrudates are dried for 16 h at 80° C. in a ventilated oven and then calcined for 4 h at 450° C.

The extrudates obtained have an SPC value of 0.26 daN/mm and an SBET of 27 m²/g, a TPV of 0.46 cm3/g with a macropore volume of 0.22 cm3/g, a mesopore volume of 0.20 cm3/g and a Dmacro=1140 nm. The material obtained has a ratio KPO3/(KPO3+SiO2)=26.1%.

TABLE 1
summary of the data from the examples.

| | | | % | | | | | | |
|---|---|---|---|---|---|---|---|---|---|
| | Source | | CsPO₃/(CsPO₃ + SiO₂) | Source of | % | % | % KH₂PO₄/ | | |
| | of | | or % | precipitated | silica | colloïdal | CsH₂PO₄ | S_{BET} | VPT_{Hg injected} |
| | K or Cs | Milling | KPO₃/(KPO₃ + SiO₂) | silica | powder | silica | sol | (m²/g) | (ml/g) |
| Example 1 | KH₂PO₄ | no | 26.17% | Siliaflash | 35.5 | 35.5 | 29 | 103 | 0.52 |
| | | | | C60 5-20 μm | | | | | |
| Example 2 | KH₂PO₄ | yes | 26.17% | Siliaflash | 35.5 | 35.5 | 29 | 96 | 0.46 |
| | | | | C60 5-20 μm | | | | | |
| Example 3 | KH₂PO₄ | no | 66.76% | Siliaflash | 56 | 15 | 70 | <1 | 0.32 |
| | | | | C60 5-20 μm | | | | | |
| Example 4 | CsH₂PO₄ | no | 33.17% | Nyasil 20 | 64.31 | 9 | 35 | 22 | 0.17 |
| Example 5 | KH₂PO₄ | yes | 26.18% | Nyasil 20 | 64.31 | 10.97 | 24.73 | 56 | 0.33 |
| Example 6 | KH₂PO₄ | yes | 26.18% | Nyasil20 | 64.31 | 10.97 | 24.73 | 37 | 0.38 |
| Example 7 | KH₂PO₄ | yes | 26.18% | Nyasil20 | 64.31 | 10.97 | 24.73 | 21 | 0.25 |
| Example 8 | KPO₃ | no | 26.10% | Nyasil20 | 36.95 | 36.95 | 26.1 | 60 | 0.34 |
| Example 9 | KPO₃ | no | 26.10% | Nyasil20 | 36.95 | 36.95 | 26.1 | 43 | 0.44 |
| Example 11 | K₂HPO₄ + | no | 26.1 | Siliaflash | 68.6 | 0 | 178 | 27 | 0.46 |
| | (NH₄)₂HPO₄ | | | C60 5-20 μm | | | K₂HPO₄ + 13.6 | | |
| | | | | | | | (NH₄)₂HPO₄ | | |

| | | | | Mean |
|---|---|---|---|---|
| | V | | | SPC of |
| | macro | Dmacro | V meso | extrudate |
| | (ml/g) | (nm) | (ml/g) | (daN/mm) |
| Example 1 | 0.19 | 1099 | 0.25 | 0.28 |
| Example 2 | 0.17 | 679 | 0.25 | 0.39 |
| Example 3 | 0.17 | 1836 | 0.01 | 0.91 |
| Example 4 | 0.01 | 808 | 0.14 | 1.9 |
| Example 5 | 0.14 | 351 | 0.15 | 0.7 |
| Example 6 | 0.18 | 310 | 0.16 | 1.05 |
| Example 7 | 0.12 | 307 | 0.07 | 2.81 |
| Example 8 | 0.06 | 260 | 0.25 | 0.85 |
| Example 9 | 0.18 | 706 | 0.23 | 1 |
| Example 11 | 0.22 | 1140 | 0.2 | 0.26 |

Example 1 illustrates the process according to the invention and shows that the material obtained can be formed into shape.

Comparison of Example 1 and of Example 2 at equivalent contents of constituent elements of the mixture shows that the milling of KH2PO4 makes it possible to obtain a final material having an improved SPC relative to the material obtained in Example 1 (0.2 daN/mm vs 0.4 daN/mm). Furthermore, milling promotes more compact granular stacking, leading to a reduction of the mean macropore diameter.

Comparison of Example 1 and of Example 3 shows that the use of a high content of KH2PO4 (70% by weight) makes it possible to obtain a material having an improved SPC relative to Example 1 (0.2 daN/mm vs 0.9 daN/mm).

Comparison of Example 2 and of Example 5, for an equivalent content of SiO2 and for a similar content of KPO3/(KPO3+SiO2) shows that the combination of milling of KH2PO4 combined with the use of a source of precipitated silica having a low particle size enables both the production of a material with an improved SPC and a lower macropore diameter making it possible to increase the density of the material (0.39 daN/mm vs 0.7 daN/mm).

Comparison of Example 3 and of Example 5 shows that the milling of KH2PO4 combined with the use of a source of precipitated silica having a small grain size makes it possible to obtain a final material having a comparable SPC but with a lower ratio KPO3/(KPO3+SiO2) in the material of Example 5 (0.9 daN/mm vs 0.7 daN/mm) (content of KPO3/(KPO3+SiO2)=26 vs KPO3/(KPO3+SiO2)=67). Less KH2PO4 precursor is thus used to obtain a comparable SPC. Better conservation of the specific surface area is also found in Example 5.

Comparison of Example 6 and of Example 5 at equivalent contents of constituent elements of the mixture shows that implementation of the hydrothermal treatment step d) at atmospheric pressure makes it possible to obtain a final material having an improved SPC in Example 6 relative to Example 5 (1.05 daN/mm vs 0.7 daN/mm).

Comparison of Example 7 and of Example 6 at equivalent contents of constituent elements of the mixture shows that implementation of the hydrothermal treatment step d) under pressure makes it possible to obtain a final material having an improved SPC in Example 7 relative to Example 6 (2.81 daN/mm vs 1.05 daN/mm).

Examples 8 and 9, at equivalent contents of constituent elements of the mixture, show the possibility of using the potassium (tripotassium) phosphate (3K+ PO43−) precursor combined with implementation of the hydrothermal treatment step d) at atmospheric pressure or under partial pressure of water to obtain a final material with a good SPC (0.85 daN/mm and 1 daN/mm).

The catalysts were tested in two experimental devices.

### SET UP A—16 Reactors Set Up Brief Description

The catalysts are tested in an Avantium device with 16 fix beds reactors in parallel. The inner quartz reactor diameter is 2 mm with a length of 560 mm. The liquid and the gas are distributed at the inlet of the reactor and mixed before introduction. The evaporation of the feed is carried out at the head of the reactor with an absorbent material (wick).

After reactor, all the products are analyzed online by gas chromatography.

### SET UP B—4 Reactors Set Up Brief Description

The catalysts were tested in an Avantium apparatus with 4 fixed bed reactors in parallel. The internal quartz reactor diameter is 2 mm or 4 mm with a length of 560 mm. The liquid and the gas are distributed at the inlet of the reactor and mixed before introduction. The evaporation of the feed is carried out at the head of the reactor with an absorbent material (wick).

After reactor, the gas phase is cooled and condensed. The liquid is collected at atmospheric pressure and at 10° C. and further analyzed by HPLC. The gas phase is analyzed online by gas chromatography.

### Example 1: Catalytic Performances in a 16 Reactors Device (SET UP A)

The catalysts are tested in the device of 16 reactors in parallel (SET UP A).

The volume of catalyst loaded is 0.2 mL. The powdered and the shaped catalysts are loaded into the tube with an internal diameter of 2 mm. The reaction is carried out in gas phase, at a temperature of 375° C. and a pressure of 10 barr. The feed is a 12 wt. % lactic acid in water. The weight hourly space velocity of lactic acid is in between 0.2 and 0.25 h-1 with a mass of catalyst loading between 120 to 200 mg.

AA yield is determined by GC analysis at 20 h run time.

TABLE 2

| | | | | | | AA |
|---|---|---|---|---|---|---|
| | | MPO3/(MPO3 + | | Bed | m | yield |
| Catalyst | Example | SiO2) | | hight | catalyst | % |
| Label | patent | (% pds.) | Shape | cm | g | molC |
| A1 | Ex 1 | 26% KPO3 | Extudates 1.6 | 55 | 130 | 83% |
| A2 | Ex 2 | 26% KPO3 | Extudates 1.6 mm | 55 | 140 | 81% |
| A3 | Ex 3 | 67% KPO3 | Extudates 1.6 mm | 55 | 184 | 78% |
| A4 | Ex 4 | 33% CsPO3 | Extudates 1.6 mm | 55 | 217 | 78% |
| A5 | Ex 5 | 26% KPO3 | Extudates 1.6 | 55 | 158 | 80% |
| A6 | Ex 6 | 26% KPO3 | Extudates 1.6 | 55 | 148 | 80% |
| A7 | Ex 8 | 26% KPO3 | Extudates 1.6 mm | 55 | 144 | 79% |
| A8 | — | 26% KPO3 | Powder 100-250 | 55 | 152 | 81% |
| | | | microns | | | |
| A9 | — | 37% CsPO3 | Powder 100-250 | 55 | 199 | 70% |
| | | | microns | | | |

Catalysts A1 to A9 are active and selective in converting lactic acid to acrylic acid, with carbon yield higher than 70%. The unloading of shaped catalysts A1 to A7 is easier than for powdered catalysts A8 and A9. Indeed, the powder is stuck to the wall of the quartz reactor. An advantage of using a shaped catalyst is the easier way to unload it.

### Example 2: Catalytic Performance in a Device Comprising 4 Reactors in Parallel (SET UP B)

The catalysts are tested in the device comprising 4 fixed bed reactors in parallel with different operating conditions from example 1: higher volume of catalyst, higher total pressure, and different analytical setup.

The volume of catalyst loaded is 1 mL. The powdered catalyst is loaded into the 4.00 mm ID tubes while the shaped catalysts are loaded into the 2 mm ID tubes. The reaction is carried out in gas phase, temperature of 375° C., a pressure of 25 barg. The feed is a 20 wt. % lactic acid in water. The liquid hourly space velocity is 1.2 h-1. The nitrogen flowrate is 25 Nml/min. The gas hourly space velocity is 3000 h-1.

The conversion of lactic acid is defined as follows:

LA conversion=100×(LA in−LA out (% pds))/LA in
With LA: LA concentration in liquid phase from feed and sample (HPLC analysis) g/l

AA yield (% mol C)=100×(AA out)/(LA in-LA out)
LA in and LA out: LA concentration in the liquid phase from feed and sample (HPLC analysis) mol C/1
AA: AA concentration in liquid phase of the sample (HPLC analysis) mol C/1

The catalytic performances are given for a TOS of 18 h.

TABLE 3

| | | | | m | LA | AA |
|---|---|---|---|---|---|---|
| | | | V | catalyst | conversion | yield |
| | | Shape | catalyst | g | % | % molC |
| A7 | 26% KPO3 | Extrudates 1.6 mm | 0.9 mL | 0.580 | 99.5 | 74 |
| A8 | 26% KPO3 | Powder | 0.9 mL | 0.690 | 99.4 | 63 |
| | | 100-250 microns | | | | |
| A4 | 33% CsPO3 | Extrudates 1.6 mm | 0.9 mL | 0.700 | 98.4 | 61 |
| AS | 39% CsPO3 | Powder | 0.9 mL | 0.9120 | 99.6 | 36 |
| | | 100-250 microns | | | | |

Catalysts A4, A7, A8 and A9 are active and selective in converting lactic acid to acrylic acid, with a carbon yield higher than 70%. The conversion of lactic acid is always above 99%.

Unloading shaped catalyst A7 and A4 is less difficult than discharging powdered catalysts A7 and A8. Powdered catalyst have the potential to adhere to the walls of the reactor. Therefore, the shaped catalyst has a distinct advantage when it comes to unloading.

1. Process for the preparation of acrylic acid comprising the steps of: i) preparing a lactic acid containing feedstock, ii) heating the feedstock to obtain a lactic acid containing vapor phase, iii) contacting the lactic acid containing vapor phase with a catalyst to create a reacted vapor phase, and iv) isolating acrylic acid from the reacted vapor phase where in the catalyst is prepared by the following process:
a) a step of mixing at least one precipitated silica powder and at least one colloidal silica sol with at least one powder of at least one potassium phosphate salt and/or at least one powder of a cesium salt in at least one solvent to obtain a mixture,
b) a step of forming the mixture obtained on conclusion of step a) into a specific shape, and
c) a step of maturing the material obtained on conclusion of step b).

2. Process according to claim 1 in which the precipitated silica powder is chosen from the commercial sources Nyasil20 (Nyacol®), Siliaflash P60 (Nyacol®), Siliaflash C60 (Silicycle®), Ultrasil VN3 GR (Evonik®), taken alone or as a mixture.

3. Process according to claim 2 in which the precipitated silica powder has a grain size of less than 10 μm, less than 5 μm, or less than 1 μm.

4. Process according to claim 2, in which the colloidal silica sols are chosen from the commercial sources Ludox (W.R. Grace Davison®), Nyacol (Nyacol Nano Technologies®, Inc. or PQ Corp®), Nalco (Nalco Chemical Company®), Ultra-Sol (RESI Inc®), NexSil (NNTI®), taken alone or as a mixture.

5. Process according to claim 4 in which the potassium phosphate salts are chosen from the following list: KH₂PO₄, KH₂P₂O₁₂, K₆P₆O₇, K₃H₂P₃O₁₀, K₄H₂P₄O₁₃, K₃P₃O₉, K₄P₄O₁₂, K₆P₆O₁₈, K₈P₈O₂₄, K₁₀P₁₀O₃₀, potassium (tripotassium) phosphate (3K⁺ PO43⁻), alone or as a mixture.

6. Process according to claim 5 in which the cesium phosphate salts are chosen from the following list: CsH₂PO₄, Cs₂H₂P₃O₁₀, Cs₄H₂P₄O₁₃, Cs₃P₃O₉, Cs₄P₄O₁₂, Cs₆P₆O₁₈, Cs₈P₈O₂₄, (CsPO₃), alone or as a mixture.

7. Process according to claim 1 in which at least one organic adjuvant chosen from cellulose derivatives, polyethylene glycols, aliphatic monocarboxylic acids, aromatic alkylated compounds, sulfonic acid salts, fatty acids, polyvinylpyrrolidone, polyvinyl alcohol, methylcellulose, polyacrylates, polymethacrylates, polyisobutene, polytetrahydrofuran, starch, polymers of polysaccharide type (such as xanthan gum), scleroglucan, derivatives of hydroxyethylcellulose or carboxymethylcellulose type, lignosulfonates and galactomannan derivatives, taken alone as a mixture, is also mixed during step a).

8. Process according to claim 6 in which said solvent is chosen from water, ethanol, alcohols and amines, and preferably said solvent is water.

9. Process according to claim 8, in which said powders of at least one potassium and/or cesium phosphate salt are milled and screened to a particle size of less than 100 μm.

10. Process according to claim 9 in which the source of precipitated silica or silica gel used in step a) has a grain size of less than 10 μm, less than 5 μm, or less than 1 μm.

11. Process according to claim 10 in which said maturation step is performed at a temperature of between 20° C. and 200° C. for a time of between 1 hour and 24 hours.

12. Process according to claim 11, in which the material formed into shape and resulting from the maturation step c) undergoes a calcining step c′) at a temperature of between 100° C. and 550° C. for a time of between 1 hour and 12 hours.

13. Process according to claim 12 in which said process comprises a step d) of hydrothermal treatment in the presence of steam, said step d) possibly being performed at atmospheric pressure or under partial pressure of water.

14. Process according to claim 5 wherein process is performed at a temperature of between 350° C. and 400° C. and the feedstock contains 15% to 85% lactic acid by weight.

15. Process according to claim 13, in which the hydrothermal treatment step d) is performed at atmospheric pressure, at a temperature of between 400° C. and 1000° C., for a time of between 30 minutes and 5 hours, the composition by volume of water in the gas in said step d) being between 5% and 100%.

16. Process according to claim 15, in which the hydrothermal treatment step d) is performed at a temperature of between 100° C. and 450° C. for a time of between 30 minutes and 4 hours and under a partial pressure of water of between 0.11 and 7.5 MPa.
